(19) United States
(12) Patent Application Publication
(10) Pub. No.: US 2026/0357350 A1
(43) Pub. Date: Nov. 12, 2026

(54) METHODS FOR DETECTION OF BREAST CANCER

(71) Applicant: BAR ILAN UNIVERSITY, Ramat Gan (IL)

(72) Inventors: Sol EFRONI, Rehovot (IL); Alona ZILBERBERG, Ramat Hasharon (IL); Hagit PHILIP, Ramla (IL); Miri GORDIN, Sitria (IL)

(73) Assignee: BAR ILAN UNIVERSITY, Ramat Gan (IL)

(21) Appl. No.: 19/121,240

(22) PCT Filed: Oct. 24, 2023
(86) PCT No.: PCT/IL2023/051098

§ 371 Date: Apr. 15, 2025

Related U.S. Application Data
(60) Provisional application No. 63/380,818, filed on Oct. 25, 2022.

(51) Int. Cl. C12Q 1/6886; G16B 40/00; G16H 50/20

(52) U.S. Cl. CPC C12Q 1/6886; G16B 40/00; G16H 50/20

(57) ABSTRACT

The presently disclosed subject matter provides methods for diagnosing breast cancer. It relates to the identification of unique T cell receptor (TCR) CDR3 clones whose frequency in the subject's TCR repertoire sequencing data is indicative of disease.

### TECHNOLOGICAL FIELD

The present disclosure is in the field of diagnostics, particularly non-invasive early detection of breast cancer.

### BACKGROUND ART

References considered to be relevant as background to the presently disclosed subject matter are listed below:
Azizi, E., et al: Single-cell map of diverse immune phenotypes in the breast tumor microenvironment. Cell, 174(5): p. 1293-1308 e36 (2018).
Beausang, J., et al: T cell receptor sequencing of early-stage breast cancer tumors identifies altered clonal structure of the T cell repertoire. Proc Natl Acad Sci USA, 114(48): p. E10409-E10417 (2017).
Benichou, J., et al: Rep-Seq: uncovering the immunological repertoire through next-generation sequencing. Immunology 135(3): 183-191 (2011).
Bolotin, D. A., et al.: MiXCR: software for comprehensive adaptive immunity profiling. Nat Methods, 12(5): p. 380-1 (2015).
Calis Jorg J. A. and Rosenberg Brad R.: Characterizing immune repertoires by high throughput sequencing: strategies and applications. Trends Immunol. 35(12): 581-590 (2014).
Dupic, T., et al.: Genesis of the alphabeta T-cell receptor. PLoS Comput Biol. 15(3): p. e1006874 (2019).
Gordin, M., et al.: Breast cancer is marked by specific, public T-cell receptor CDR3 regions shared by mice and humans. PLoS Comput Biol. 17(1): p. e1008486 (2021).
Kivioja, T., et al.: Counting absolute numbers of molecules using unique molecular identifiers. Nat Methods, 9(1): p. 72-4 (2012).
Madi, A., et al.: T cell receptor repertoires of mice and humans are clustered in similarity networks around conserved public CDR3 sequences. eLife, 6 (2017).
Matsuda T., et al: TCR sequencing analysis of cancer tissues and tumor draining lymph nodes in colorectal cancer patients. OncoImmunology, 8(6): e1588085 (2019).
Page D., et al: Deep sequencing of T-cell receptor DNA as a biomarker of clonally expanded TILs in breast cancer after immunotherapy. Cancer Immunol Res, 4(10): p. 835-844 (2016).
Rubelt F., et al: Adaptive immune receptor repertoire community recommendations for sharing immune-repertoire sequencing data. Nature Immunology 18:1274-1278 (2017).
Sherwood A. M., et al: Tumor-infiltrating lymphocytes in colorectal tumors display a diversity of T cell receptor sequences that differ from the T cells in adjacent mucosal tissue. Cancer Immunology, Immunotherapy 62: 1453-1461 (2013).
Sun G., et al.: Association of the characteristics of B- and T-cell repertoires with papillary thyroid carcinoma. Oncol. Lett. 16(2): 1584-1592 (2018).
Tickotsky, N., et al.: McPAS-TCR: a manually curated catalogue of pathology-associated T cell receptor sequences. Bioinformatics, 33(18): p. 2924-2929 (2017).
Tomczak, K., et al.: The Cancer Genome Atlas (TCGA): an immeasurable source of knowledge. Contemp. Oncol (Pozn) 19(1A): p. A68-77 (2015).
Vander Heiden, J. A., et al.: pRESTO: a toolkit for processing high throughput sequencing raw reads of lymphocyte receptor repertoires. Bioinformatics, 30(13): p. 1930-2 (2014).
Wang, T., et al: The different T-cell receptor répertoires in breast cancer tumors, draining lymph nodes, and adjacent tissues. Cancer Immunol Res, 5(2): p. 148-156 (2017).
Yaari, G. and S. H. Kleinstein: practical guidelines for B-cell receptor repertoire sequencing analysis. Genome Med, 7:121 (2015).
Zhang Y., et al: Deep single-cell RNA sequencing data of individual T cells from treatment-naïve colorectal cancer patients. Nature Scientific Data, 6:131 (2019).

Acknowledgement of the above references herein is not to be inferred as meaning that these are in any way relevant to the patentability of the presently disclosed subject matter.

### BACKGROUND

Cancer is a leading cause of morbidity and mortality world-wide. Thus, it is vitally important to come up with an inexpensive and easy-to-use technology for early-stage screening, since early detection and treatment were found to be the best way to decrease deaths due to cancer.

The population of T lymphocytes collectively comprises a huge repertoire of receptors (TCRs) that are highly diverse in their antigen-binding sites. Each receptor is composed of two distinct subunit chains (primarily α/β chains). The TCR antigen binding surface is formed by the variable region of each chain, which is encoded by somatic V (D) J DNA recombination, and constant gene segments (e.g. constant region TCR alpha/beta) [Calis and Rosenberg (2014)]. The total diversity established through these molecular mechanisms is substantial, with the estimated theoretical numbers of TCRs exceeding 10\hat{ }¹⁸ [Sun et al (2018)].

Despite the astronomical numbers of possible CDR3 nucleotide recombinations [Dupic et al (2019)], prevalent CDR3 segments appear across species in mice and humans [Madi et al (2017)].

High-throughput sequencing of B and T cell receptors, also termed “Repertoire sequencing” (Rep-Seq) [Benichou et al (2011)] or AIRR-seq [Rubelt et al (2017)], is often applied in studies of adaptive immunity. Other works focused on tumor infiltrating lymphocytes (TIL) found both in tumors and the surrounding tissue, to investigate the T-cell repertoire (TCR) [Sherwood et al (2013)] or sequencing tumor draining lymph nodes [Matsuda et al (2019)] to differentiate between metastasis and non-malign tissue. Recent works [Zhang et al (2019)] also took advantage of single cell RNA sequencing (scRNAseq) to further explore TIL of CRC patients.

Gordin et al (2021) discloses that breast cancer is marked by specific, public T-cell receptor CDR3 regions shared by mice and humans.

### GENERAL DESCRIPTION

In a first of its aspects, the present invention provides a method for diagnosing breast cancer in a subject, the method comprising:
a. obtaining T cell receptor repertoire sequencing data (TCR Rep-Seq) of said subject;
b. measuring the frequency within the TCR Rep-Seq of a set of CDR3 clones associated with breast cancer; and
c. based on the frequency data of the set of CDR3 clones associated with breast cancer, calculating an estimated probability for the presence or absence of breast cancer in the subject.

In one embodiment, obtaining TCR Rep-Seq data of said subject comprises the steps of:
a. obtaining at least one sample of said subject, wherein said sample comprises T cells;
b. optionally, isolating the T cells from said sample;
c. extracting RNA from said sample or said isolated T cells;
d. constructing TCR-seq libraries;
e. subjecting said TCR-seq libraries to high-throughput sequencing; and
f. analyzing the sequencing data to produce a TCR repertoire comprising CDR3 clone sequences and their abundance in each sample.

In one embodiment, said step of constructing TCR-seq libraries comprises performing amplification and next generation sequencing (NGS) of the RNA extracted from said sample or said isolated T cells.

In one embodiment, said sample comprising T cells is selected from a group consisting of a blood sample, saliva, a lymph node biopsy, and a breast cancer biopsy.

In one embodiment, said T cells are isolated peripheral blood mononuclear cells (PBMCs).

In one embodiment, the set of CDR3 clones associated with breast cancer is identified using a machine learning classification model.

In one embodiment, the machine learning classification model is developed by a method comprising:
a. presenting the model with a training set comprising TCR repertoire sequencing data of known breast cancer patients and known healthy subjects;
b. training the model to identify CDR3 sequences that are characteristic to and discriminate between breast cancer patients and healthy subjects.

In some embodiments, said machine learning model is selected from a group consisting of a decision tree algorithm, Linear Discriminant Analysis (LDA), Bayesian classifiers, random forests, support vector machines (SVM), logistic regression models, elastic net regression models, k-nearest neighbors (kNN), neural networks, and ensemble models.

In one embodiment, said set of CDR3 clones comprises at least one, at least two, or at least three CDR3 clones associated with breast cancer.

In one embodiment, said set of CDR3 clones consists of three CDR3 clones associated with breast cancer.

In one embodiment, the CDR3 clones associated with breast cancer comprise sequences having at least 92% homology, or 100% homology, with SEQ ID NO: 1, SEQ ID NO: 2, and SEQ ID NO: 3.

In one embodiment, calculating an estimated probability for the presence or absence of breast cancer comprises introducing the frequency of each clone in the set to a machine learning model which determines an estimated probability for the presence or absence of breast cancer in the subject.

In one embodiment, the machine learning model is a decision tree algorithm.

In one embodiment, the method of the invention is a method for early detection of breast cancer.

In one embodiment, the method of the invention is a method for screening the population for detection of breast cancer.

In one embodiment, said subject is a human subject.

In another aspect, the present invention provides a computer software product, comprising a computer-readable medium in which program instructions are stored, which instructions, when read by a data processor, configure the data processor to (1) receive T cell receptor (TCR) repertoire sequencing data of a subject, and to (2) execute the method of the invention.

In another aspect, the present invention provides a system for diagnosing breast cancer in a subject, comprising: an input utility for receiving T cell receptor (TCR) repertoire sequencing data of a subject; and a data processor configured for analyzing said data for executing the method of the invention.

### BRIEF DESCRIPTION OF THE DRAWINGS

To better understand the subject matter that is disclosed herein and to exemplify how it may be carried out in practice, embodiments will now be described, by way of non-limiting example only, with reference to the accompanying drawings, in which:

FIG. 1A-1B are graphs showing the number of unique CDR3 amino acid (AA) sequences (A) and their copies (B) in beta chain in breast cancer patients (BRCA) and control samples.

FIG. 2A-2B are graphs showing the number of unique CDR3 AA sequences (A) and their copies (B) in alpha chain in breast cancer patients and control samples.

FIG. 3A-3B are graphs showing clonality (A) and diversity (B) in beta chains in breast cancer patients (BRCA) and control samples.

FIG. 4A-4B are graphs showing clonality (A) and diversity (B) in alpha chains in breast cancer patients (BRCA) and control samples.

FIG. 5A-5B are graphs showing unique clones (AA sequences) in beta chain (A) and in alpha chain (B) in breast cancer patients and control samples.

FIG. 6A-6B are graphs showing diversity in alpha chain (A) and in beta chain (B) in breast cancer patients and control samples.

FIG. 7A-7B are graphs showing clonality in alpha chain (A) and in beta chain (B) in breast cancer patients and control samples.

FIG. 8 is a schematic representation showing a general description of a confusion matrix of the algorithm's success.

FIG. 9 is a ROC curve of a breast cancer dataset classifying breast cancer via spectral embeddings. The graph shows the mean ROC. AUC=0.91±0.08.

### DETAILED DESCRIPTION OF EMBODIMENTS

The present disclosure is based on the development of a unique machine learning platform that facilitates the identification of T cell receptor (TCR) CDR3 clones that appear at the onset and during disease, for example breast cancer-specific clones, and can therefore be used as biomarkers for early detection.

A TCR is a disulfide-linked heterodimeric protein consisting of two variable chains expressed as part of a complex with the invariant CD3 chain molecules. A TCR is found on the surface of T cells and is responsible for recognizing antigens as peptides bound to major histocompatibility complex (MHC) molecules. A TCR may comprise an α chain and a β chain (encoded by TRA and TRB, respectively). Alternatively, a TCR may comprise a γ chain and a δ chain (encoded by TRG and TRD, respectively).

Each chain of a TCR comprises two extracellular domains: a variable region and a constant region. The constant region is proximal to the cell membrane, followed by a transmembrane domain and a short cytoplasmic tail (i.e., an intracellular domain). The variable region binds to the peptide/MHC complex. The variable region of both chains each has three complementarity determining regions (CDRs).

T cell receptor repertoires can be exploited to serve as a seismograph to report early changes in many physical conditions and pathologies, for example cancer.

A person's TCR repertoire, which can be revealed for example by sequencing and analyzing peripheral blood samples containing T cells, can be indicative of hyperproliferative conditions (e.g., tumors) in the body. It is also postulated that the immune system detects tumors early enough for TCR repertoire technologies to be informative early on regarding tumor status, long before such tumors present clinical manifestations. Accordingly, TCR repertoire data from peripheral blood is a pragmatic way to search for early signs of cancer inflicting a minimal burden on the patient, possibly with enhanced sensitivity. However, the commonly used characterization of the TCR repertoire is not sufficient to distinguish between a healthy and a diseased status of the patient and can therefore not be relied upon for diagnostic purposes.

A machine learning model can receive sequence information of the TCR repertoire as an input and can calculate a characteristic value which can be indicative of the presence of breast cancer. The machine learning model can be trained based on the training dataset. In some implementations, the machine learning model can be provided with the TCR Rep-Seq info of breast cancer samples and healthy subjects samples in the training set as input and can calculate a characteristic value (also referred to as “score”) for each sample.

Accordingly, to identify the subtle changes in the TCR repertoire that indicate a hyperproliferative state, a classifier was developed in accordance with the invention using a decision tree algorithm based on quantified levels of TCR clones.

The present invention therefore provides elements within the TCR repertoires that are associated with a pathological condition, e.g., breast cancer.

Furthermore, in accordance with the present disclosure, the breast cancer diagnosis can be performed using a sample comprising T cells, e.g., a blood sample.

Analysis of the TCR repertoire behavior can therefore be used for early detection of cancer through a simple and inexpensive blood test.

As shown in Example 1 below, unique T-cell receptor (TCR) repertoires associated with breast cancer were identified.

Accordingly, the present invention concerns a method for diagnosing a disease (e.g., breast cancer) in a subject, the method comprising:
a. obtaining T cell receptor repertoire sequencing data (TCR Rep-Seq) of said subject;
b. measuring the frequency within the TCR Rep-Seq of a set of CDR3 clones associated with breast cancer; and
c. based on the frequency data of the set of CDR3 clones associated with breast cancer, calculating an estimated probability for the presence or absence of breast cancer in the subject.

As used herein, the term “disease” refers to any condition or disorder that damages or interferes with the normal function of a cell, tissue, or organ. An example of a disease is neoplasm.

As used herein, the term “neoplasm” refers to a disease characterized by the pathological proliferation of a cell or tissue and its subsequent migration to or invasion of other tissues or organs. Neoplasm growth is typically uncontrolled and progressive, and occurs under conditions that would not elicit, or would cause cessation of, multiplication of normal cells. Neoplasms can affect a variety of cell types, tissues, or organs, including but not limited to breast cancer.

In a specific embodiment, the term “breast cancer” refers to HER2-positive breast cancer. HER2-positive breast cancer is a breast cancer that tests positive for human epidermal growth factor receptor 2 (HER2).

As used herein, the term “subject” refers to a human patient. In an embodiment, the term refers to a female human patient.

The terms “abundance” and “frequency” are used interchangeably herein and refer to the proportional occurrence, namely, the number of times that a clone appears in the TCR repertoire of the subject, divided by the total number of clones in the TCR repertoire of the subject.

In an embodiment, the estimated probability is calculated by introducing the frequency data into a machine learning tool to create a model that predicts the class to which every sample is classified. This prediction is referred to herein as the “estimated probability”. The classification is based both on the identity of the clones and their frequency. In a specific embodiment, the machine learning model is a decision tree algorithm. In the creation of the model, the data is repeatedly divided into subsets of clones based on their frequencies. This division is performed in a way that minimizes impurity, which means it aims to create subsets of clones where the class labels are as pure as possible. The estimated probability is computed as the fraction of training examples in the leaf node that belongs to that class. When a prediction is made, the input data is passed through the tree structure, starting at the root node, and moving down the branches. At each internal node, a decision is made based on the frequency of the specific clone, and the data is directed to the next node. This process continues until the data reaches a leaf node, where a specific class label is assigned. The assigned class label is determined by the majority class of training examples that reached that leaf, i.e., according to the estimated probability.

As used herein, “CDRs” are defined as the complementarity determining region amino acid sequences of a TCR, which are the hypervariable regions of TCR α-chain and β-chain. Generally, a TCR comprises at least three CDRs in the α-chain variable region and at least three CDRs in the β-chain variable region. CDRs provide most contact residues for the binding of the TCR to the antigen or epitope. The CDRs regions may be delineated for example using the Kabat system, the Chothia numbering system, the AbM numbering system or the IMGT numbering system.

For example, in one embodiment, the CDR3 clones associated with breast cancer have a sequence with at least 92% homology with a sequence selected from a group consisting of CASSLGRTEAFF (SEQ ID NO: 1), CASSLARHNEQFF (SEQ ID NO: 2), and CASSLGQGGTEAFF (SEQ ID NO: 3). In a specific embodiment, the CDR3 clones associated with breast cancer have a sequence selected from a group consisting of CASSLGRTEAFF (SEQ ID NO: 1), CASSLARHNEQFF (SEQ ID NO: 2), and CASSLGQGGTEAFF (SEQ ID NO: 3).

In certain embodiments, if the frequency of CASSLGRTEAFF (SEQ ID NO: 1) is above 20⁻⁶ then the subject is diagnosed as having breast cancer. In other embodiments, if the frequency of CASSLGRTEAFF (SEQ ID NO: 1) is below 20⁻⁶ and the frequency of CASSLARHNEQFF (SEQ ID NO: 2) is below 31⁻⁶ then the subject is diagnosed as having breast cancer. In other embodiments, if the frequency of CASSLGRTEAFF (SEQ ID NO: 1) is below 20⁻⁶, the frequency of CASSLARHNEQFF (SEQ ID NO: 2) is above 31⁻⁶, and the frequency of CASSLGQGGTEAFF (SEQ ID NO: 3) is below 31⁻⁶ than the subject is defined as healthy (namely, the subject is diagnosed as not having breast cancer).

As used herein the term “estimated probability” refers to a probability that is at least 85%, at least 90%, or at least 92% that the subject suffers from the disease. For example, and as shown in the Examples below, a decision tree classifier composed from 3 TCR CDR3 sequences (e.g., CASSLGRTEAFF (SEQ ID NO: 1), CASSLARHNEQFF (SEQ ID NO: 2), and CASSLGQGGTEAFF (SEQ ID NO: 3) can distinguish breast cancer patients from the control samples with 92% success rate.

The terms “T cell receptor (TCR) repertoire sequencing data” and “TCR Rep-Seq” are used interchangeably herein and refer to the sequencing information of the entire T cell receptor repertoire of a subject.

In an embodiment, the TCR repertoire sequencing data is obtained by the following steps:
(a) obtaining at least one sample of said subject, wherein said sample comprises T cells;
(b) optionally, isolating the T cells from said sample;
(c) extracting RNA from the sample or from said isolated T cells;
(d) constructing TCR-seq libraries; and
(e) subjecting said TCR-seq libraries to high-throughput sequencing; and
(f) analyzing the sequencing data to produce a TCR repertoire comprising CDR3 clone sequences and their abundance in each sample.

In one embodiment, the sample is a peripheral blood mononuclear cells (PBMC) sample, which has a high T cell content. In such case, no further isolation of T cells is required prior to RNA extraction.

In other embodiments, the sample is peripheral blood, lymph node biopsy, saliva, or a tumor biopsy. In such case, the method optionally comprises isolation of T cells from the sample prior to RNA extraction. As used herein the term “isolated T cells”, refers to a T cell fraction (comprising one or more cells) that is separated from the original sample using methods well known in the art.

A standard peripheral blood sample, having a volume of e.g., about 5 ml, comprises about 10⁶ cells, about 60% of which are T cells. In accordance with the invention the sample comprises at least 1000 T cells.

Producing TCR repertoire data of a subject is performed by TCR-Seq (T-cell Receptor Sequencing). TCR-Seq utilizes the unique nature of the TCR as a molecular barcode for recognizing T cells.

The TCR are membrane proteins constructed as heterodimers made up of predominantly α and β chains (or more rarely δ and γ chains) and consist of a variable region and a constant region. Variable regions are produced through a process called VDJ recombination, which results in unique amino acid sequences for α, β, and γ chains. The result is that each TCR is unique and recognizes a specific antigen.

CDRs are a part of the TCR. CDR3 is the most variable of the CDRs, and as such, it is used as the “barcode region” to identify unique T cell populations, as it is highly unlikely for two T cells to have the same CDR3 sequence unless they came from the same parental T cell, namely a clone. When an antigen is properly presented in the body, the few T cells that recognize that antigen rapidly divide and differentiate into effector T-cells through a process called clonal expansion, which retains the TCR sequence (including the CDR3 sequence) that originally recognized the antigen.

In accordance with the invention, TCR-Seq is performed on cell populations obtained from samples comprising T cells, e.g., blood (particularly peripheral blood), peripheral blood mononuclear cell (PBMC) populations isolated from the peripheral blood of a tested subject, lymph node biopsy, saliva, or tumor biopsy. TCR-Seq allows the determination of the frequency of a specific clone (CDR3 sequence) within the entire TCR repertoire. The preparation of the TCR-Rep Seq can be performed from freshly harvested T cells (e.g., in the form of a PBMC sample or in the form of isolated T cells from any type of sample as described above) or from frozen samples.

RNA is extracted from the cells using methods known in the art (e.g., using a commercial kit, e.g., as described in the Examples).

Construction of TCR-seq libraries is performed by selection and amplification, e.g., using polymerase chain amplification (PCR), of the CDR3 region which characterizes T cells within a population.

The amplification can be performed using a commercial kit, e.g., as described in the Examples. A non-limiting example is the SMARTer TCR profiling kit (Takara) which allows to capture complete V(D)J variable regions of TCR alpha and beta chains and to exponentially amplify this segment by PCR. As a result, a library of transcripts is generated which are then subjected to sequencing, e.g., by next generation sequencing (NGS). As used herein the term “Next Generation Sequencing (NGS)” refers to a high throughput sequencing technology, or deep sequencing, as described for example in Attia and Saeed (Next Generat. Sequenc. & Applic. 2016, S1:006 DOI: 10.4172/2469-9853.S1-006). An exemplary method is provided in the Examples below.

Sequencing can be performed for example on an Illumina sequencer (e.g., Illumina sequencer) using the 300-cycle Nextseq 500/550 Mid Output Kit v2.5 with pair-end, 2×150 base pair reads, or the 600-cycle Miseq reagent kit v3 (Illumina) with pair-end, 2×300 base pair reads which enables the capturing of the CDR3 domain of each TCR α/β transcript.

In an embodiment, the output files from the Illumina sequencer are analyzed by various dedicated software tools. Exemplary software tools include but are not limited to VDJdb and McPas-TCR (Tickotsky et al., 2017), the Cogent NGS Immune Profiler Software designed to align sequences, incorporating an inherent MiXCR, and to fix PCR errors; Immunarch (an open-source tool), a specialized suite developed for the analysis of TCR-seq or BCR-seq (for B cell analysis); or QC validation (e.g., “STAR”). Alignment of the sequences can be performed using MiXCR, a universal tool for extracting CDR3 (and joining) sequences from fastq data (Bolotin et al, 2015) or IMGT tools. In some embodiments, MiXCR/IMGT are used without previously employing Cogent. In other embodiments, Cogent is employed prior to using the alignment tools, e.g., MiXCR or IMGT. MiXCR performs paired end read merging and extracts human or animal TCR clonotypes providing corrections for erroneous sequences introduced by NGS (Vander Heiden, J. A., et al., (2014); Kivioja, T., et al., (2012); Yaari, G. and S. H. Kleinstein (2015).

Optionally prior to the sequence alignment step, the raw sequences are cleaned and preprocessed to remove noise, artifacts, and irrelevant information.

In an embodiment, sequences associated with breast cancer are identified using a predictive multi-feature machine learning classification model comprising the following steps:
Obtaining CDR3 RNA sequence data (the TCR-repertoire) of multiple samples from healthy subjects (also referred to herein as “donors”) and from patients with breast cancer. The TCR repertoire data optionally also comprises amino acid sequences and corresponding metadata, e.g., disease stage, subject's age, and body mass index (BMI). The TCR repertoire sequencing data may be obtained from public sources such as The Cancer Genome Atlas (TCGA) (Tomczak et al (2015) or published human TCR breast cancer datasets: as described for example in Wang et al (2017), Page et al (2016), and Beausang et al (2017). Published single-cell RNA-seq TCR data of breast cancer patients, e.g., as described in Azizi et al (2018), can also be used.
Filtering and keeping only the shared sequences in the dataset.
Model Training and Hyperparameter Tuning: Training a machine learning algorithm or model, such as, but not limited to, Random forests, support vector machines, or deep neural networks (DNN), and choosing optimal hyperparameters for each model. Overfitting is prevented by splitting the training dataset into a sub-train set and a validation set.
A decision tree algorithm (also referred to herein as a “decision tree classifier”) composed of 3 TCR CDR3 sequences was the best performer with an average AUC of 0.91 (as indicated by the mean AUC in FIG. 9). This decision tree classifier was able to distinguish the breast cancer patients from the control samples with about 92% accuracy.
Model Fitting: Fitting the model on the entire training set while optimizing its parameters for better performance.
Model Evaluation: Evaluating the trained model's performance using the test dataset, utilizing metrics such as, but not limited to, accuracy or precision.
Model Choosing: Choosing a model with the best performance on the testing dataset.
Interpretation: Interpreting the model's results and selecting features that contribute most to its predictions.
Fine-tuning: Adjusting the model based on insights gained from the interpretation to enhance its accuracy and reliability.
Deployment: deploying the model to analyze new Rep-seq data and make diagnostic predictions.
Continuous Monitoring and Updating: Continuously monitor the model's performance and update it as new data becomes available or as needed.

In an embodiment the method of the invention is used for screening the general population or for screening subjects in risk groups for developing breast cancer. In an embodiment the method of the invention is used for early detection of cancer. As used herein the term “early detection” refers to detection prior to appearance of clinical manifestations, or detection at early stages of clinical manifestations.

Embodiments described herein may include both hardware and software elements. In an embodiment, the present invention is implemented in software.

Embodiments may include a computer program product accessible from a computer-usable or computer-readable medium providing program code for use by or in connection with a computer or any instruction execution system. A computer-usable or computer-readable medium may include any apparatus that stores, communicates, propagates, or transports the program for use by or in connection with the instructions execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid-state memory, magnetic tape, a removable computer diskette, a random-access memory (RAM), a read-only memory (ROM), a rigid magnetic disk and an optic disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein, namely to (1) receive T cell receptor (TCR) repertoire sequencing data of a subject, and to (2) execute the method of the invention as described herein.

The system of the invention may also be embodied in a computer-readable storage medium, configured with a computer program, where the storage medium so configured causes a computer to operate in a specific and predefined manner to perform the functions described herein.

Unless defined otherwise, all technical and scientific terms used herein have the meaning commonly understood by a person skilled in the art to which this invention belongs.

As used herein, the term “about” means within an acceptable error range for the value as determined by one of ordinary skill in the art, which will depend in part on how the value is measured or determined, i.e., the limitations of the measurement system. For example, “about” can mean a range of up to 5%, more preferably up to 2.5%, and more preferably still up to 1% of a given value.

### EXAMPLES

### Example 1: Early Detection of Breast Cancer Using a Blood Sample

PBMCs of HER-2 positive breast cancer patients (N=48) and healthy women donors (N=48) were used for the analysis. The samples were collected in the past and were kept at the Institutional Sample Database (Tissue Bank), Ichilov.

The frozen PBMCs samples were thawed, and their total RNA content was extracted using the RNeasy mini kit.

The obtained RNA was then evaluated for its integrity value (RIN) to better standardize RNA quality. Next, RNA concentrations were determined by qubit, which provides an accurate method for the quantitation of low abundance RNA samples. A fixed total RNA concentration of 250 ng from each sample was subjected to the SMARTer TCR profiling kit (Takara); first single cDNA strand was synthetized at the first round of reverse transcription, using poly T primer for the 3′ end and a smarter oligo (Switching Mechanism at 5′ End of RNA Template) for the 5′ ends, catalyzed by MMLV-derived SMARTScibe Reverse Transcriptase. By employing 5′ RACE-like approach, the SMARTer TCR profiling kit allows to capture complete V(D)J variable regions of TCR alpha and beta chains and in the following step each fragment is exponentially increased by PCR. On each dsDNA fragment, dedicated adapters containing flow cell binding sites, P5 and P7, were inserted to allow the enriched library fragments to attach to the cell flow surface.

The TCR sequencing library was size-selected and purified using AMPure XP beads.

The generated libraries were measured for their DNA concentration by qubit and assessed for their size by TapeStation (the libraries may also be assessed using Bioanalyzer). These parameters enable calculation of library molarity (final pool molarity range from 2 to 4 nM) to pool together 20 libraries for a single flow cell sequencing by preserving an equal representation of each single library at the final pool.

TCR sequencing was performed on an Illumina Miseq sequencer using the 600-cycle Miseq reagent kit v3 (Illumina) with pair-end, 2×300 base pair reads.

The data generated from each pooling went through a bioinformatics pipeline including QC validation (e.g., “STAR”) and MiXCR, a universal tool for extracting CDR3 (and joining) sequences from fastq data.

### Results

Table 1 presents the total count of copies and unique nucleotides (Nt) or amino acid (Aa) sequences across all sequenced samples. S1-S40-Breast cancer patients, HER2 positive; S41-S106-healthy donors.

TABLE 1

| Sample | Copies | Unique Nt | Unique Aa | Copies | Unique Nt | Unique Aa |
|---|---|---|---|---|---|---|
| Id | Alpha | Alpha | Alpha | Beta | Beta | Beta |
| 1_S12 | 126622 | 57702 | 51944 | 58543 | 40608 | 39908 |
| 1_S13 | 111374 | 20666 | 19057 | 53532 | 27578 | 6991 |
| 1_S16 | 83334 | 31281 | 28786 | 73092 | 39446 | 38625 |
| 1_S17 | 71117 | 23526 | 21945 | 108873 | 40085 | 39131 |
| 1_S18 | 15209 | 6669 | 6455 | 24784 | 11495 | 11315 |
| 1_S19 | 41818 | 14858 | 14077 | 49633 | 19757 | 19322 |
| 1_S2 | 30634 | 13045 | 12423 | 54289 | 25860 | 25355 |
| 1_S20 | 34559 | 11348 | 10787 | 47418 | 18537 | 18121 |
| 1_S3 | 25797 | 5834 | 5515 | 61154 | 15553 | 14987 |
| 1_S4 | 25207 | 9553 | 9172 | 57690 | 20048 | 19655 |
| 1_S5 | 138987 | 37103 | 33937 | 191100 | 60884 | 58859 |
| 1_S6 | 141875 | 56130 | 50095 | 239491 | 106286 | 101314 |
| 1_S7 | 147195 | 35276 | 32360 | 211009 | 58793 | 56980 |
| 1_S8 | 83206 | 27349 | 25437 | 166910 | 63698 | 61420 |
| 1_S9 | 25932 | 7037 | 6791 | 97114 | 24740 | 24097 |
| 2_S22 | 24636 | 10259 | 9832 | 41846 | 23834 | 23490 |
| 2_S31 | 16543 | 10876 | 10474 | 33586 | 23050 | 22740 |
| 2_S32 | 71926 | 21384 | 20171 | 127201 | 48201 | 46794 |
| 2_S33 | 6398 | 3762 | 3687 | 6676 | 4193 | 4159 |
| 2_S34 | 5491 | 3962 | 3891 | 11220 | 8300 | 8236 |
| 2_S35 | 6286 | 3504 | 3434 | 9965 | 5708 | 5663 |
| 2_S36 | 5104 | 2414 | 2358 | 4081 | 2111 | 2084 |
| 2_S37 | 2244 | 1653 | 1636 | 5438 | 3943 | 3919 |
| 2_S38 | 28671 | 14852 | 14126 | 54698 | 27455 | 26944 |
| 2_S39 | 5317 | 3496 | 3432 | 9290 | 5443 | 5403 |
| 2_S40 | 5293 | 3225 | 3182 | 13840 | 7901 | 7829 |
| 2_S23 | 2588 | 1719 | 1695 | 2991 | 2097 | 2081 |
| 2_S1 | 4933 | 1471 | 1434 | 10019 | 3391 | 3331 |
| 2_S11 | 99654 | 27173 | 25248 | 128859 | 34106 | 33428 |
| 2_S15 | 128297 | 38105 | 34901 | 54339 | 34981 | 34353 |
| 2_S24 | 12137 | 4572 | 4466 | 17620 | 9164 | 9056 |
| 2_S25 | 48074 | 21317 | 20006 | 66239 | 33240 | 32552 |
| 2_S26 | 14806 | 6242 | 6068 | 23200 | 9988 | 9859 |
| 2_S27 | 4967 | 3045 | 2983 | 10590 | 6740 | 6685 |
| 2_S28 | 3131 | 2128 | 2111 | 3224 | 2497 | 2486 |
| 2_S29 | 30670 | 19299 | 18324 | 53060 | 32261 | 31719 |
| 2_S30 | 7718 | 6007 | 5862 | 14309 | 11073 | 10994 |
| 3_S41 | 61865 | 29038 | 27038 | 50647 | 32605 | 32112 |
| 3_S52 | 22812 | 9878 | 9467 | 20770 | 10603 | 10532 |
| 3_S53 | 9554 | 5077 | 4927 | 18036 | 10133 | 10034 |
| 3_S54 | 3324 | 2026 | 2000 | 4065 | 2651 | 2634 |
| 3_S55 | 24204 | 15509 | 14774 | 38534 | 27835 | 27421 |
| 3_S56 | 187032 | 15728 | 14937 | 102562 | 22832 | 22354 |
| 3_S57 | 96749 | 17614 | 16706 | 175153 | 61463 | 59679 |
| 3_S58 | 37857 | 15583 | 14686 | 43217 | 22454 | 22064 |
| 3_S59 | 72576 | 34602 | 32038 | 110142 | 59158 | 57548 |
| 3_S60 | 32074 | 10020 | 9660 | 75589 | 21275 | 20886 |
| 3_S42 | 98801 | 12035 | 11520 | 51859 | 9819 | 9678 |
| 3_S43 | 171140 | 65234 | 58422 | 106909 | 61218 | 59685 |
| 3_S44 | 172434 | 53958 | 48451 | 107701 | 60962 | 59253 |
| 3_S45 | 32727 | 10135 | 9593 | 18962 | 10592 | 10455 |
| 3_S46 | 4149 | 2147 | 2106 | 1668 | 1235 | 1229 |
| 3_S49 | 66891 | 32249 | 29901 | 31133 | 22588 | 22301 |
| 3_S48 | 53723 | 23156 | 21624 | 57690 | 35686 | 35012 |
| 3_S51 | 64589 | 21827 | 20408 | 52711 | 26321 | 25754 |
| 4_S64 | 338788 | 100919 | 86604 | 269578 | 139474 | 132750 |
| 4_S65 | 386465 | 110554 | 94784 | 298926 | 147601 | 141178 |
| 4_S66 | 226458 | 38313 | 34776 | 232082 | 70240 | 67836 |
| 4_S67 | 577896 | 170985 | 139713 | 459419 | 230998 | 217266 |
| 4_S68 | 169861 | 33874 | 30820 | 159103 | 48894 | 47274 |
| 4_S69 | 169722 | 63680 | 56663 | 135036 | 78397 | 75876 |
| 4_S70 | 67387 | 20755 | 19379 | 71260 | 39893 | 38986 |
| 4_S71 | 163836 | 59735 | 53024 | 98738 | 59338 | 57918 |
| 4_S72 | 157297 | 49385 | 44950 | 81987 | 44568 | 43686 |
| 4_S73 | 51527 | 15775 | 14995 | 49261 | 20775 | 20434 |
| 4_S74 | 59485 | 18472 | 17302 | 76777 | 32685 | 31957 |
| 4_S75 | 91802 | 41676 | 38123 | 30393 | 24432 | 24161 |
| 4_S76 | 34502 | 18623 | 17601 | 83762 | 51743 | 50572 |
| 4_S63 | 47127 | 21079 | 19935 | 81609 | 37248 | 36485 |
| 5_S80 | 61468 | 23269 | 21633 | 77917 | 39306 | 38405 |
| 5_S89 | 9513 | 6312 | 6150 | 18275 | 12131 | 12001 |
| 5_S90 | 12510 | 7035 | 6838 | 20425 | 11586 | 11431 |
| 5_S91 | 132780 | 30537 | 28143 | 94828 | 29418 | 28758 |
| 5_S92 | 77124 | 46648 | 41871 | 42742 | 32448 | 31765 |
| 5_S93 | 109305 | 64307 | 57451 | 62917 | 48323 | 47205 |
| 5_S94 | 213787 | 107385 | 92011 | 106748 | 83553 | 81242 |
| 5_S95 | 80205 | 42688 | 38992 | 48158 | 35461 | 34906 |
| 5_S96 | 78784 | 36482 | 33618 | 46839 | 30159 | 29699 |
| 5_S97 | 54900 | 16794 | 15795 | 61815 | 20080 | 19740 |
| 5_S98 | 157220 | 76853 | 68139 | 93457 | 61124 | 59595 |
| 5_S81 | 32431 | 9906 | 9436 | 32168 | 12325 | 12131 |
| 5_S82 | 23755 | 8407 | 8051 | 28516 | 13993 | 13765 |
| 5_S83 | 27046 | 9057 | 8604 | 26897 | 12608 | 12374 |
| 5_S84 | 17573 | 11574 | 11097 | 18203 | 11945 | 11841 |
| 5_S85 | 97940 | 31368 | 28745 | 84273 | 47296 | 46165 |
| 5_S86 | 15624 | 6618 | 6404 | 16173 | 10396 | 10271 |
| 5_S87 | 12203 | 6564 | 6367 | 4251 | 3430 | 3411 |
| 5_S88 | 35128 | 16540 | 15627 | 12146 | 9491 | 9412 |
| 6_S99 | 223558 | 58965 | 52366 | 148886 | 64243 | 62451 |
| 6_S100 | 182144 | 35221 | 32285 | 130331 | 34408 | 33720 |
| 6_S101 | 43575 | 13511 | 12879 | 38401 | 11017 | 10820 |
| 6_S102 | 116075 | 33022 | 30505 | 73643 | 30209 | 29614 |
| 6_S103 | 54722 | 24449 | 22870 | 32942 | 18709 | 18437 |
| 6_S104 | 220542 | 71885 | 63201 | 119776 | 58125 | 56584 |
| 6_S105 | 221082 | 83438 | 72857 | 114474 | 61134 | 59663 |
| 6_S106 | 50874 | 18910 | 17829 | 25786 | 11481 | 11316 |

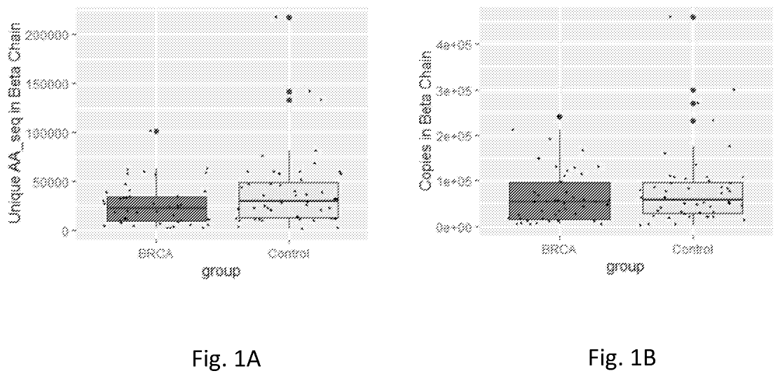

FIG. 1 shows the number of unique CDR3 sequences (A) and their copies (B) in breast cancer patients and control samples in beta chains.

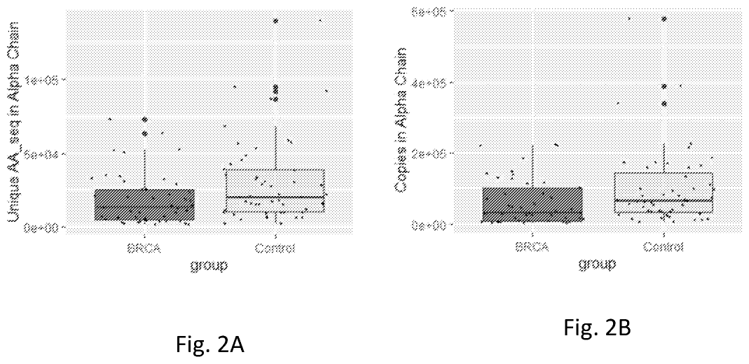

FIG. 2 shows the number of unique CDR3 sequences (A) and their copies (B) in breast cancer patients and control samples in alpha chains.

Variation in sample sizes accounts for differences in the unique sequences of the sample. Clonality and diversity measures were used to explore the TCR repertoire of these samples. Clonality is defined as (1—Pielou's evenness) where diversity is defined as the inverse Simpson index. Diversity describes the number of unique sequences and their proportional evenness, whereas clonality describes the shape of the distribution of proportional abundances and ranges from 0 to 1. Clonality values approaching 1 indicate an increasingly asymmetric distribution in which a few clones are present at high frequencies.

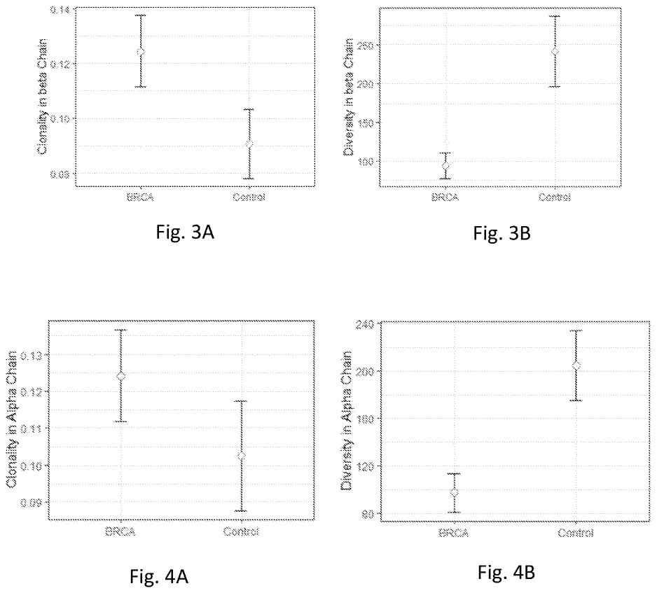

FIG. 3 shows the Clonality (A) and diversity (B) in beta chains.

FIG. 4 shows the Clonality (A) and diversity (B) in alpha chains.

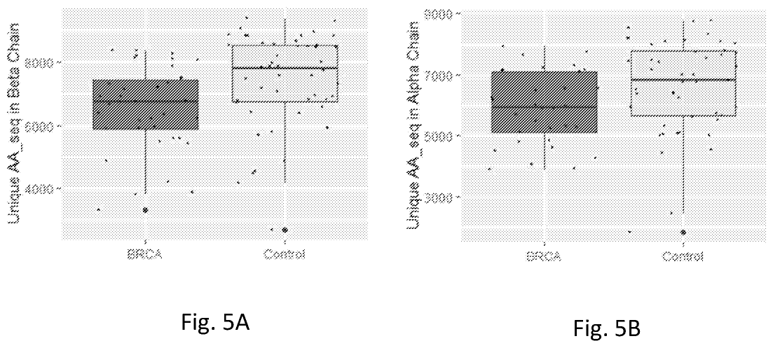

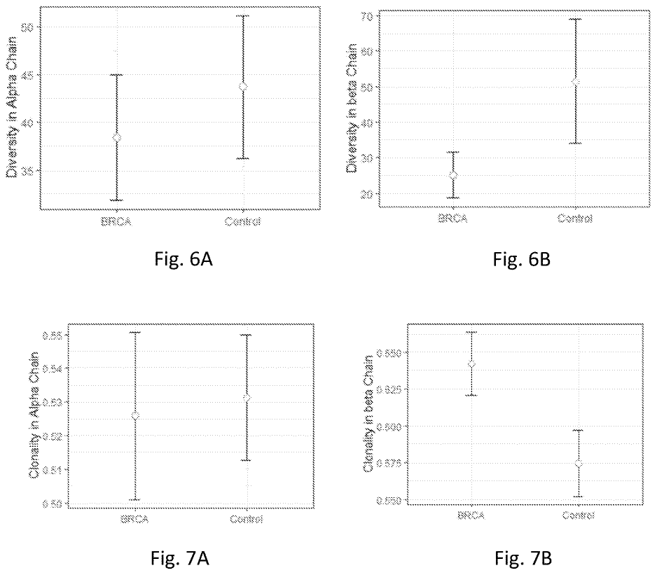

To avoid bias originating from different sample sizes, 10,000 sequences were subsampled, as the number of clones in the smallest samples. The numbers of unique clones, diversity and clonality in alpha and beta chains in the sub sampled sequences are shown in FIGS. 5, 6, and 7, respectively.

To build an algorithm that uses the levels of TCR clones for the classification of breast cancer patients and control samples, different machine learning algorithms were used over these data. A decision tree classifier composed of 3 TCR CDR3 sequences was able to distinguish the breast cancer patients from the control samples with about 92% success rate.

The TCR CDR3 sequences that were identified are:

(SEQ ID NO: 1)
CASSLGRTEAFF
(SEQ ID NO: 2)
CASSLARHNEQFF
(SEQ ID NO: 3)
CASSLGQGGTEAFF

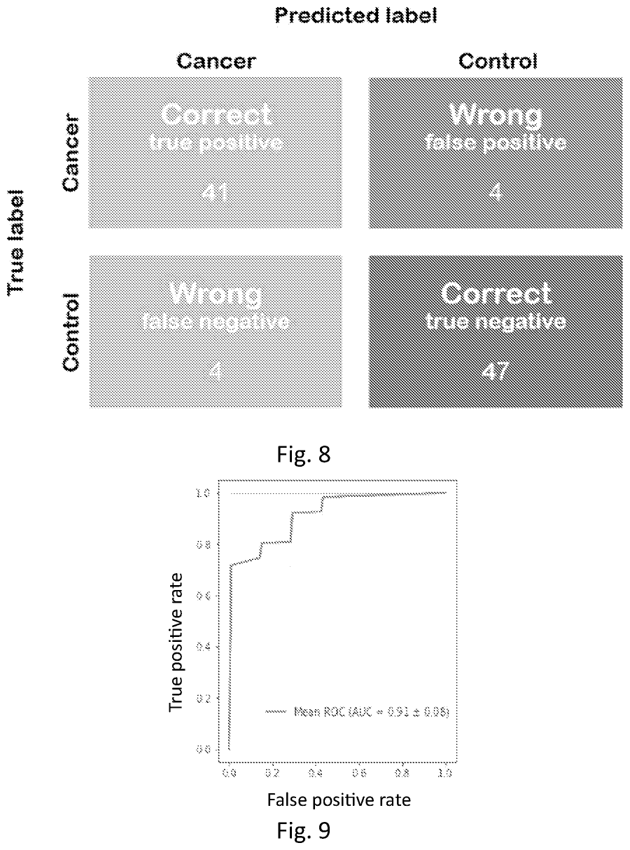

A confusion matrix of algorithm's success is shown in FIG. 8, representing a combined train and test data.

A total number of 96 samples were analyzed. 41 were found to be “Correct—true positive”, 47 “Correct—true negative”, 4 “false positive”, and 4 “false negative”.

Accuracy was measured according to the following formula:

41(“Correct—true positive”)+47(“Correct—true negative”) divided by the total number of samples:41(“Correct—true positive”)+47(“Correct—true negative”)+4(“false positive”)+4(“false negative”)×100=91.6%.

False positive rate was measured according to the following formula:

4(“false positive”) divided by 47(“Correct—true negative”)+4(“false positive”)×100=7.8%.

False negative rate was measured according to the following formula:

4(“false negative”) divided by 41(“Correct—true positive”)+4(“false negative”)×100=8.88%.

Sensitivity (recall) was measured according to the following formula:

41(“Correct—true positive”) divided by 41(“Correct—true positive”)+4(“false negative”)×100=91.1%.

Specificity was measured according to the following formula:

47(“Correct—true negative”) divided by 47(“Correct—true negative”)+4(“false positive”)×100=92.1%.

Precision was measured according to the following formula:

41(“Correct—true positive”) divided by 41(“Correct—true positive”)+4(“false positive”)×100=91.1%.

To show the stability of the model, AUC-ROC curves were used to measure the predictive power of sample correct classification. A ROC curve (receiver operating characteristic curve) is a tool showing the performance of a classification model at all classification thresholds. This curve uses two parameters: True Positive Rate (TPR) and False Positive Rate (FPR). A ROC curve plots TPR vs. FPR at different classification thresholds. Lowering the classification threshold classifies more items as positive, thus increasing both False Positives and True Positives. AUC stands for “Area under the Curve.” That is, AUC measures the entire two-dimensional area under the entire ROC curve from (0,0) to (1,1). FIG. 9 shows the success of the method in classifying the set of 50 women with breast cancer versus 50 healthy women referred to above. An AUC of 0.91 indicates that blood samples from women with or without breast cancer can be stratified with very high success rates.

The CDR3 sequence graph and its mathematical representation were used as an adjacency matrix, as substrate for Spectral Graph Embedding, with which it is possible to describe each repertoire using the set of eigenvectors of the adjacency matrix. This approach eventually provides dimensionality reduction using the 10 most represented eigenvectors, thereby translating each repertoire to 20-dimensions. These dimensions (features) are then used for machine learning.

Spectral Graph Embedding is a dimensionality reduction technique used in machine learning and data analysis. It involves representing the nodes of a graph in a lower-dimensional space while preserving certain structural properties of the original graph. With respect to the present invention, CDR3 sequences are gathered by their similarity editing distance which is determined by comparing CDR3 amino acids sequences looking for amino acid substitutions in a specific position.

Spectral Graph Embedding relies on spectral analysis, where the eigenvalues and eigenvectors of the graph's Laplacian matrix are computed. In spectral graph embedding, the graph is transformed into a spectral domain, and each node is mapped to a point in this spectral space. The goal is to position nodes in such a way that nodes that are structurally similar in the original graph are still close to each other in the lower-dimensional space. One common application of spectral graph embedding is in clustering and visualization. By embedding the graph's nodes in a lower-dimensional space, it becomes easier to identify groups or clusters of nodes based on their structural similarities. Techniques like t-SNE are popular methods for spectral graph embedding.

1.-18. (canceled)

19. A method for diagnosing breast cancer in a subject, the method comprising:
a. obtaining T cell receptor repertoire sequencing data (TCR Rep-Seq) of said subject;
b. measuring the frequency within the TCR Rep-Seq of a set of CDR3 clones associated with breast cancer; and
c. based on the frequency data of the set of CDR3 clones associated with breast cancer, calculating an estimated probability for the presence or absence of breast cancer in the subject.

20. The method of claim 19 wherein obtaining TCR Rep-Seq data of said subject comprises the steps of:
a. obtaining at least one sample of said subject, wherein said sample comprises T cells;
b. optionally, isolating the T cells from said sample;
c. extracting RNA from said sample or said isolated T cells;
d. constructing TCR-seq libraries;
e. subjecting said TCR-seq libraries to high-throughput sequencing; and
f. analyzing the sequencing data to produce a TCR repertoire comprising CDR3 clone sequences and their abundance in each sample.

21. The method of claim 20 wherein said step of constructing TCR-seq libraries comprises performing amplification and next generation sequencing (NGS) of the RNA extracted from said sample or said isolated T cells.

22. The method of claim 20 wherein said sample comprising T cells is selected from a group consisting of a blood sample, saliva, a lymph node biopsy, and a breast cancer biopsy.

23. The method of claim 20 wherein said T cells are isolated peripheral blood mononuclear cells (PBMCs).

24. The method of claim 19, wherein the set of CDR3 clones associated with breast cancer is identified using a machine learning classification model.

25. The method of claim 24 wherein the machine learning classification model is developed by a method comprising:
g. presenting the model with a training set comprising TCR repertoire sequencing data of known breast cancer patients and known healthy subjects;
h. training the model to identify CDR3 sequences that are characteristic to and discriminate between breast cancer patients and healthy subjects.

26. The method of claim 24, wherein said machine learning model is selected from a group consisting of a decision tree algorithm, Linear Discriminant Analysis (LDA), Bayesian classifiers, random forests, support vector machines (SVM), logistic regression models, elastic net regression models, k-nearest neighbors (kNN), neural networks, and ensemble models.

27. The method of claim 19, wherein said set of CDR3 clones comprises at least one, at least two, or at least three CDR3 clones associated with breast cancer.

28. The method of claim 27, wherein said set of CDR3 clones consists of three CDR3 clones associated with breast cancer.

29. The method of claim 28 wherein the CDR3 clones associated with breast cancer comprise sequences having at least 92% homology, or 100% homology with SEQ ID NO: 1, SEQ ID NO: 2, and SEQ ID NO: 3.

30. The method of claim 19 wherein calculating an estimated probability for the presence or absence of breast cancer comprises introducing the frequency of each clone in the set to a machine learning model which determines an estimated probability for the presence or absence of breast cancer in the subject.

31. The method of claim 30 wherein the machine learning model is a decision tree algorithm.

32. The method of claim 19, wherein said method is a method for early detection of breast cancer.

33. The method of claim 19, wherein said method is a method for screening the population for detection of breast cancer.

32. The method of claim 19 wherein said subject is a human subject.

33. A computer software product, comprising a computer-readable medium in which program instructions are stored, which instructions, when read by a data processor, configure the data processor to (1) receive T cell receptor (TCR) repertoire sequencing data of a subject, and to (2) execute the method according to claim 19.

34. A system for diagnosing breast cancer in a subject, comprising: an input utility for receiving T cell receptor (TCR) repertoire sequencing data of a subject; and a data processor configured for analyzing said data for executing the method according to claim 19.


SEQUENCE LISTING (3 sequences)

SEQ ID NO: 1 - protein, 12 aa, Homo sapiens
CASSLGRTEAFF

SEQ ID NO: 2 - protein, 13 aa, Homo sapiens
CASSLARHNEQFF

SEQ ID NO: 3 - protein, 14 aa, Homo sapiens
CASSLGQGGTEAFF